(19) United States
(12) Patent Application Publication
(10) Pub. No.: US 2026/0034159 A1
(43) Pub. Date: Feb. 5, 2026

(54) METHODS FOR PRODUCING CYCLIC DINUCLEOTIDES

(71) Applicant: Aldevron, LLC, Fargo, ND (US)

(72) Inventors: DENIS ARUTYUNOV, Fargo, ND (US); TODD MICHAEL VANNELLI, Fargo, ND (US)

(21) Appl. No.: 19/099,727

(22) PCT Filed: Aug. 10, 2023
(86) PCT No.: PCT/US2023/072024

§ 371 Date: Jan. 29, 2025

Related U.S. Application Data
(60) Provisional application No. 63/371,063, filed on Aug. 10, 2022.

(51) Int. Cl. A61K 31/7084; C12P 19/36

(52) U.S. Cl. CPC A61K 31/7084; C12P 19/36

(57) ABSTRACT

The present disclosure provides a method of producing cyclic dinucleotides (CDNs) on a commercial scale. Also provided are pharmaceutical compositions comprising a purified CDN preparation and use thereof to stimulate the immune system in a subject.

### CROSS-REFERENCE TO RELATED APPLICATION

This application claims priority from U.S. Provisional Application 63/371,063, filed Aug. 10, 2022, the entire disclosure of which is incorporated herein by reference.

### BACKGROUND OF THE INVENTION

Cyclic dinucleotides (CDNs) are signaling molecules that control important biological functions in bacteria relating to biofilm development, motility, cell shape and cycle, and pathogenicity. In mammalian cells, CDNs play a role in innate immunity (Jenal et al., Nat Rev Microbiol. (2017) 15:271-84; Krasteva and Sondermann, Nat Chem Biol. (2017) 13:350-9). Cyclic GMP-AMP (cGAMP) is a CDN composed of one adenine monophosphate (AMP) and one guanine monophosphate (GMP) connected by two phosphodiester bonds. One such cGAMP, 2′,3′-cGAMP (2′-3′-cyclic GMP-AMP), is an endogenous cGAMP in mammalian cells. It is a potent inducer of interferon-β (IFNβ) and is produced in mammalian cells in response to DNA in the cytoplasm (Zhang et al., Mol Cell. (2013) 25:51(2):226-35).

CDNs are currently manufactured by chemical or enzymatic synthesis (Romling et al., Microbiol Mol Biol Rev. (2013) 77:1-52). Both processes have their own drawbacks. Chemical synthesis is time-consuming and is not environmentally sound (Gaffney et al., Org Lett. (2010) 12:3269-71; Gaffney and Jones, Curr Protoc Nucleic Acid Chem. (2012) Chapter 14, Unit 14.8.1-7; Schwede et al., Handb Exp Pharmacol. (2017) 238:359-84). Enzymatic synthesis of CDNs using dinucleotide cyclases (DNCs) requires precursor GTP, which is costly (Spehr et al., Appl Biochem Biotechnol. (2011) 165:761-75). Microbes such as E. coli cells also have been used to generate CDNs (Lv et al., Front Microbiol. (2019) 10:2111). However, the methods known to date use low bacterial cell densities and do not allow for production of CDNs at a large scale.

Accordingly, there remains a need for a more efficient and economical method of producing CDNs at a commercial scale.

### SUMMARY OF THE INVENTION

The present disclosure provides a method of producing cyclic dinucleotides (CDNs), comprising: (a) incubating CDN-producing recombinant E. coli cells in a culture medium in a fermenter to produce a desired amount of CDN; (b) removing the cells from the cell culture medium to obtain a cell-free culture medium containing CDN; (c) filtering the cell-free culture medium to remove cell debris; (d) reducing the salt concentration of the filtered culture medium through dilution; (e) isolating CDN from the mixture of step (d) through a column that does not comprise affinity binding; and (f) purifying the isolated CDN through nanofiltration.

In some embodiments, the recombinant E. coli cells are incubated in a minimal fermentation medium in the fermenter. In further embodiments, the minimal fermentation medium is supplemented and comprises: M9 minimal salts; 0.8-3% glucose; 5-10 mM MgSO4; 0.1-0.3 mM CaCl2); and 0.01-0.03 mM ferrous sulfate.

In some embodiments, the recombinant E. coli cells comprise a codon-optimized version of a mouse cyclic guanosine monophosphate (GMP)-adenosine monophosphate (AMP) synthase (mcGAS) gene.

In some embodiments, the recombinant E. coli cells contain (i) extra copies of one or more of E. coli argU, ileY, leuW, and proL tRNA genes, optionally wherein said extra copies are codon bias-adjusted; and/or (ii) a plasmid bearing a constitutively active lacI repressor gene. In certain embodiments, the mcGAS gene is expressed in the recombinant E. coli cells as part of a small ubiquitin-like modifier (SUMO) fusion protein. In certain embodiments, the E. coli cells are of a BL21-CodonPlus (DE3)-RIPL strain (Agilent) or a HI-Control BL21(DE3) (Lucigen) strain, or another derivative of BL21 or BL21(DE3) strain.

In some embodiments, the recombinant E. coli cells are grown for about 36-72, or about 36-60, optionally about 48-56, hours in the fermenter to produce CDN.

In some embodiments, the fermenter has a culture volume of 1 L to 1000 L.

In some embodiments, between steps (b) and (c) the method comprises freezing and thawing the cell-free culture medium containing CDN.

In some embodiments, step (b) comprises lysing the cultured E. coli cells.

In some embodiments, step (c) comprises using a 0.2 μm filter.

In some embodiments, wherein between steps (d) and (e) the method comprises subjecting the diluted mixture through weak anion-exchange resin or molecular weight cut-off (MWCO) filtration to remove impurities. In further embodiments, the MWCO filtration uses a 1 or 3 kDa cut-off filter.

In some embodiments, step (e) comprises: contacting the diluted mixture containing CDN with a solid support comprising strong anion-exchange resin (e.g., Q Sepharose™ or QAE Sephadex®), wherein CDN in the mixture binds to the solid support; washing the solid support with a washing solution to remove biological materials other than bound CDN, and preferentially eluting the bound CDN from the solid support with an elution solution to obtain a substantially pure CDN preparation. In further embodiments, the washing solution comprises a neutral buffer, optionally comprising 20 mM Tris-HCl, pH 7.2. In further embodiments, the elution solution comprises 20 mM Tris-HCl, pH 7.2, and 100-200 mM NaCl.

In some embodiments, step (f) comprises concentrating said CDN using tangential flow nanofiltration and/or direct flow nanofiltration.

In some embodiments, the CDN preparation is filtered to remove endotoxin.

In some embodiments, the CDN is 2′,3′-cGAMP; c-di-GMP; or 3′,3′-cGAMP.

The present disclosure also provides a pharmaceutical composition comprising a purified CDN obtained by the production method herein.

In another aspect, the present disclosure provides a method of stimulating the immune system in a subject in need thereof, comprising administering to the subject an effective amount of the present CDN-containing pharmaceutical composition. Also provided are pharmaceutical compositions for use in a method of stimulating the immune system in a subject in need thereof. The present disclosure also provides the use of a purified CDN obtained by the production method herein in the manufacture of a medicament for use in a method of stimulating the immune system in a subject in need thereof. In some embodiments, the subject has cancer or an infection.

Other features, objectives, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTION OF THE FIGURES

FIG. 1 is panel of graphs showing the results of an HPLC detection of a 2′,3′-cGAMP standard in water. The top panel shows a control run with molecular biology grade (MBG) water. The remaining panels display serial dilutions (using 0.05, 0.025, and 0.0125 mg/ml of 2′,3′-cGAMP analyte in MBG water).

FIG. 2 is a panel of graphs showing the results of an HPLC detection of 2′,3′-cGAMP secreted into the minimal media by two different E. coli clones with recombinant cGAMP synthetase (cGAS) following shake flask growth. The top panel represents E. coli clone 1, where cGAS was expressed using 0.1 mM IPTG. The second panel from the top represents E. coli clone 2, where cGAS was expressed using 0.1 mM IPTG. The third panel from the top represents E. coli clone 1, where cGAS was expressed using 0.5 mM IPTG. The fourth panel from the top represents E. coli clone 2, where cGAS was expressed using 0.5 mM IPTG. The bottom panel shows the standard 2′,3′-cGAMP analyte run.

FIG. 3 is a panel of graphs showing a 2′,3′-cGAMP fermentation trial to determine the time course of maximum 2′,3′-cGAMP production. The panels show 2′,3′-cGAMP production at 20, 48, 56, and 72 hours (starting from the top panel). The third panel from the top (56 h) shows maximum yield of 2′,3′-cGAMP.

FIG. 4 is a panel of graphs showing the presence of 2′,3′-cGAMP by HPLC produced during shake flask growth. The top panel shows the water run (control). The two middle panels show the appearance of a peak with a retention time that matches 2′,3′-cGAMP. In this trial, the mixture (referred to as starting material “SM”) from the upstream process was diluted in water 5×, and was then applied to Q Sepharose™ column, washed, and eluted. A flow-through step was carried out to confirm that most of the analyte (2′,3′-cGAMP) was retained (bottom panel), and indeed most was retained with only about 10% cGMAP flowing through.

FIG. 5 is a panel of graphs showing a purification trial after fermentation, the resulting mixture (referred to as starting material “SM”) was diluted in water 10×, and was then applied to Q Sepharose™ column, washed, and eluted. The middle panel confirms the presence of 2′,3′-cGAMP in the eluate. The eluate comprises 50 mM NaCl using 20 mM Tris-HCl buffer at pH 7.2. A flow-through step was carried out to confirm that most of the analyte was retained (bottom panel), and indeed, the analyte 2′,3′-cGMAP was retained (did not show up on flow-through), while the rest of the contaminants showed up on flow-through.

FIG. 6 is a panel of graphs showing the presence of 2′,3′-cGAMP in tangential flow filtration (TFF) retentate. The picture shows a TFF device used to concentrate 2′,3′-cGAMP.

FIG. 7 is a picture showing a direct flow filtration device, which was used to concentrate 2′,3′-cGAMP.

FIG. 8 is a schematic diagram showing the production of recombinant 2′,3′-cGAMP.

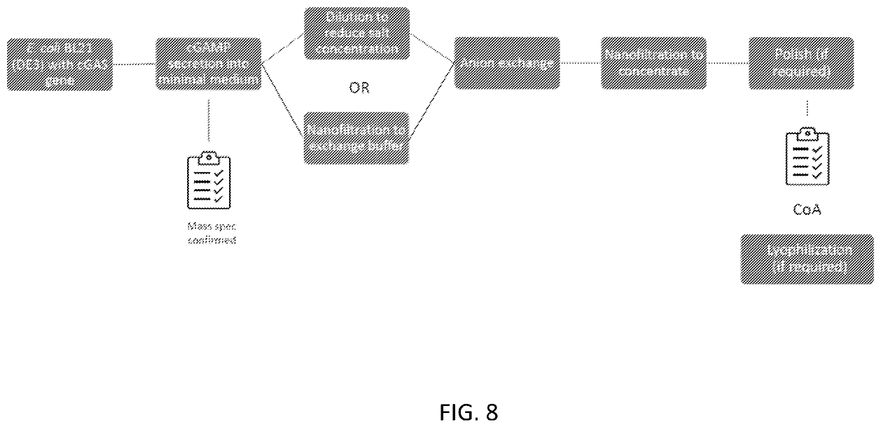

### DETAILED DESCRIPTION OF THE INVENTION

The present disclosure provides an improved process of producing CDNs (e.g., cGAMP such as 2′,3′-cGAMP and 3′,3′-cGAMP; and c-di-GMP) on a commercial scale. The process involves using fermentation of recombinant E. coli cells to obtain high bacterial cell densities and greater production of CDNs, followed by purification by a highly scalable method that does not require affinity chromatography using immobilized cGAMP receptor as originally described by Lv et al. (Front Microbiol. (2019) 10:2111). Prior methods involving affinity chromatography may be problematic for both technical and regulatory reasons. Further, prior methods generate CDNs with high levels of endotoxin (e.g., >2,000 EU/mg) and host cell proteins (e.g., about 7%). The present method produces highly purified CDN with minimal or no detectable endotoxin (e.g., <2,000, <1,000, <500, <100, <50, <10, or <1 EU/mg) and minimal or no detectable host cell protein contamination (e.g., <7%, <6%, <5%, <4%, <3%, <2% or <1%).

In some embodiments, a substantially pure CDN preparation herein contains no more than 7% (e.g., no more than 6, 5, 4, 3, 2, or 1%) impurities such as plasmid or genomic DNA, RNA, proteins, and endotoxin. In some embodiments, a substantially pure CDN solution is 95-99.99% pure.

2′,3′-cGAMP (CAS No. 1441190-66-4) is an endogenous cGAMP in mammalian cells. 2′,3′-cGAMP binds to STING with a high affinity and is a potent inducer of IFNβ. This cGAMP has the following structure:

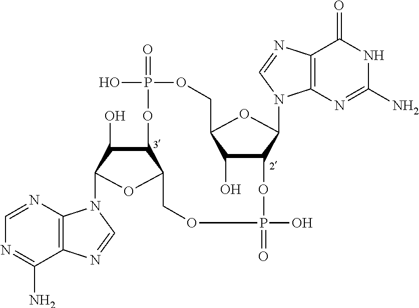

3′,3′-cGAMP (CAS No. 849214-04-6) functions as an endogenous second messenger in metazoans and triggers interferon production in response to cytosolic DNA. This cGAMP activates stimulator of interferon genes (STING), which activates a signaling cascade leading to the production of type I interferons and other immune mediators. The cGAMP has the following structure:

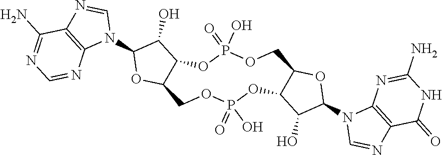 [SMILES: Nc1nc2c(ncn2[C@@H]2O[C@@H]3COP(=O)(O)O[C@H]4[C@@H](O)[C@H](n5cnc6c(N)ncnc65)O[C@@H]4COP(=O)(O)O[C@H]3[C@H]2O)c(=O)[nH]1]

c-di-GMP (cyclic-di-GMP; CAS No. 61093-23-0) is a STING agonist and a bacterial second messenger that coordinates different aspects of bacterial growth and behavior, including motility, virulence, biofilm formation, and cell cycle progression. C-di-GMP has anti-cancer cell proliferation activity and also induces elevated CD4 receptor expression and cell cycle arrest. Cyclic-di-GMP can be used in cancer research. It has the following structure:

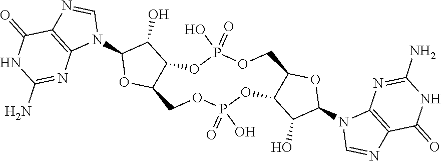 [SMILES: Nc1nc2c(ncn2[C@@H]2O[C@@H]3COP(=O)(O)O[C@H]4[C@@H](O)[C@H](n5cnc6c(=O)[nH]c(N)nc65)O[C@@H]4COP(=O)(O)O[C@H]3[C@H]2O)c(=O)[nH]1]

### I. Cyclic Dinucleotide Production

The present CDN production method uses recombinant microbial cells (e.g., E. coli cells) that have been transformed with a gene encoding a full-length murine cyclic guanosine monophosphate (GMP)-adenosine monophosphate (AMP) synthase (cGAS). The recombinant cells overexpress the murine cGAS (mcGAS), resulting in the production of CDNs, in particular 2′,3′-cGAMP. The CDNs in the cell culture media are then isolated and purified using a method that can be readily scaled up for commercial production.

### A. Production of CDNs

In some embodiments, the microbial cells used herein are bacterial cells, such as recombinant E. coli cells, that are capable of producing CDNs such as cGAMP. In some embodiments, the E. coli strain used for producing a cGAMP such as 2′,3′-cGAMP is a BL21 competent strain. BL21 competent E. coli is a widely used non-T7 expression E. coli strain and is suitable for transformation and protein expression; this strain does not express the T7 RNA polymerase.

In some embodiments, the E. coli strain is a BL21(DE3) strain. This strain contains the lambda DE3 prophage that carries the gene for T7 RNA polymerase under the control of a lacUV5 promoter, allowing expression of the T7 RNA polymerase to be induced with IPTG. BL21(DE3) is an E. coli B strain and does not contain the Lon protease (La). It is also deficient in the outer membrane protease OmpT. The lack of these two key proteases reduces degradation of heterologous proteins expressed in the cells. DE3 strains of E. coli are suitable for production of protein from target genes cloned in pET vectors by induction with IPTG.

In particular embodiments, the E. coli strain is HI-Control® BL21(DE3) chemically competent E. coli (e.g., Lucigen, Biosearch Technologies). This strain induces high-level protein expression from T7 promoters with tight control over leaky expression. The HI-Control® strain is based on E. coli JOG (DH10B) and BL21(DE3) strains; it harbors a plasmid bearing a constitutive lacI repressor gene, which provides tight control over promoters containing the lacO operator. In other particular embodiments, the E. coli strain is a HMS174(DE3) strain (e.g., EMD Millipore). This strain provides the recA mutation in a K-12 background and may stabilize certain target genes whose products may cause the loss of the DE3 prophage.

In some embodiments, the E. coli strains has one or more of the following characteristics: contain extra copies of rare E. coli argU (AGA, AGG), ileY (AUA), leuW (CUA), and/or proL (CCC) tRNA genes, which may correct codon bias and dramatically improves expression of heterologous sequences from other organisms; induce high-level protein expression from promoters with tight control over leaky expression; contain a plasmid bearing a constitutive lacI repressor gene, which may provide tight control over promoters containing the lacO operator; and achieve high efficiency transformation.

In particular embodiments, the E. coli strain is a BL21-CodonPlus® (DE3)-RIL strain (Agilent Technologies). This strain contains extra copies of the rare argU, ileY, and leuW tRNA genes. The strain may also be a BL21-CodonPlus® (DE3)-RIPL strain (Agilent Technologies), which additionally carries extra copies of the proL tRNA gene.

Other bacterial strains similar to those described above may also be used.

The E. coli strains used herein are capable of high-level expression of a cGAS transgene (e.g., a mammalian such as mcGAS gene). The DNA sequence of mcGAS is available at NCBI under Gene ID. 214763. To improve expression of the gene, the gene sequence may be codon-optimized for E. coli expression. In some embodiments, the E. coli cells are transformed with an mcGAS gene, where the mcGAS is expressed as a small ubiquitin-like modifier (SUMO) fusion protein in the E. coli cells. SUMO as an N-terminal fusion partner enhances functional protein production in prokaryotic and eukaryotic expression systems (Panavas et al., Methods Mol Biol. (2009) 497:303-17). In some embodiments, the SUMO fusion protein is expressed from a pETite N-His SUMO Kan vector (e.g., Lucigen, and NovoPro Bioscience).

In particular embodiments, the bacterial cells used herein are capable of producing and secreting 2′,3′-cGAMP. In some embodiments, the 2′,3′-cGAMP produced is about 100-200 mg/L (e.g., 150 mg/L) or higher.

In other embodiments, the bacterial cells used herein are capable of producing c-di-GMP or 3′,3′-cGAMP.

In particular embodiments, the bacterial cell may be fermented in a minimal fermentation medium into which the cell secretes increased amounts of cGAMP such as 2′,3′-cGAMP. A minimal fermentation medium is a bacterial culture medium that contains the minimal nutrients for bacterial cells to grow, and typically contains only salts and nitrogen. A minimal fermentation medium may be supplemented as needed with glucose, amino acids, and/or vitamins. One exemplary minimal fermentation medium is M9 minimal medium (e.g., Millipore Sigma). M9 minimal medium comprises M9 minimal salts, which include potassium phosphate, sodium phosphate, sodium chloride, and ammonium chloride (e.g., Millipore Sigma; 5× M9 minimal salts—15 g/L KH₂PO₄, 34 g/L Na₂HPO₄·7H₂O, 2.5 g/L NaCl, and 5.0 g/L NH₄Cl). In some embodiments, the culture medium used herein is M9 minimal medium supplemented with glucose and additional salts such as magnesium sulfate, calcium chloride, and/or ferrous sulfate. For example, the culture medium may comprise M9 minimal salts, 0.8-3% glucose, 5-10 mM MgSO₄, 0.1-0.3 mM CaCl₂), and 0.01-0.03 FeSO₄.

In some embodiments, the bacterial cell undergoes fermentation for up to 4 weeks in about 10 to 100 L runs to produce about 15 to 100 grams of CDN (e.g., 2′,3′-cGAMP). In some embodiments, at least about 100 grams of CDN are produced. In further embodiments, about 100-200, 200-400, 400-800, 800-1,000, or 1,000-2,000 grams of CDN are produced. In some embodiments, the bacterial cell undergoes fermentation for up to 4 weeks in up to 500 L, 600 L, 700 L, 800 L, 900 L, 1,000 L, or 5,000 L of fermentation medium. In some embodiments, the bacterial cell undergoes fermentation for 2 to 4 weeks. In some embodiments, the bacterial cells are cultured for one week or less. In some embodiments, the bacterial cells are cultured for one, two, three, four, five, six, or seven days. In some embodiments, the bacterial cells are cultured for about 20-72 hours, e.g., about 36-60 hours. In further embodiments, the bacterial cells are cultured for about 48-60 hours. In certain embodiments, the bacterial cells are cultured for about 56 hours. The length of fermentation and volumes may be adjusted as necessary to maximize CDN yield.

B. Removing Cells and Debris from Culture Medium

At the end of the culture period, the culture medium is harvested by removing the cells, by, e.g., centrifugation. In some embodiments, the bacterial cells in the fermenter are lysed once a desired level of bacterial cell density has been reached. After the cells are lysed, cell debris may be removed by first using DNase, RNase, and a proteinase (e.g., protease K), followed by centrifugation and/or filtration (e.g., with a 0.2 μm filter). After that, the CDN may be isolated from the supernatant.

In some embodiments, the cell culture medium is subjected to several freeze/thaw cycles to denature proteins so as to facilitate removal proteins and other contaminants present in the medium. In some embodiments, the freezing step may be carried out at about −5° C., −10° C., −15° C., −20° C., −25° C., −30° C., or −35° C., or down to, e.g., −80° C. Precipitated matters including denatured proteins and impurities attached thereto may be removed by centrifugation and/or filtration (e.g., with a 0.2 μm filter).

### C. Reduction of Salt Concentration

The cell-free culture medium containing CDNs may then be diluted to reduce salt concentrations. Salt concentration may be reduced through dilution with water to arrive at a diluted mixture. For example, the cell-free culture medium may be diluted with water up to 20-fold (e.g., up to 10-fold or up to 5-fold).

### D. Removal of Additional Contaminants

In some embodiments, between the salt reduction step and the downstream CDN isolation step discussed below, the method herein comprises subjecting the diluted CDN mixture through weak anion-exchange resin or filtration to further remove impurities. Doing so can help eliminate contaminants and interfering materials from the diluted mixture. In particular embodiments, the diluted CDN mixture is adsorbed onto weak anion-exchange resin such as diethylaminoethyl cellulose (DEAE-C). DEAE-C separates proteins that have faintly differing charges. Like all anion exchangers, the resin carries a positive charge that interacts favorably with negative charges. The positive charge of DEAE-C is due to a protonated amine group. Other weak anion exchangers are known in the art. See, e.g., Kumar, Pranav, “Fundamentals and Techniques of Biophysics and Molecular Biology,” 2018, New Delhi: Pathfinder Publication.

In some embodiments, the diluted CDN mixture is incubated with the DEAE resin for a period of about 1 to about 10 hours, e.g., about 3, 4, 5, 6, or more hours, at room temperature on a stirrer; the volume of resin may be equal or similar to the volume of the pre-diluted CDN mixture. The CDN analyte does not bind to the weak-anion exchange resin; only contaminants do. The diluted mixture and resin are then filtered (e.g., by using a 0.2 μm filter).

In some embodiments, between the salt reduction step and the downstream CDN isolation step discussed below, the method comprises subjecting the diluted CDN mixture through a molecular weight cut-off (MWCO) filter to further reduce contaminants, including interfering materials. In particular embodiments, the MWCO filter is a 1 kDa, 2 kDa, or 3 kDa cut-off filter. MWCO filters are commercially available, and any MWCO filter that fits the size criteria as per the invention requirements may be used.

### E. Isolation of CDN

In some embodiments, a CDN is isolated from the diluted mixture by adsorbing the CDN mixture to resin that does not involve affinity binding (e.g., binding to a CDN receptor). In some embodiments, the resin is an anion-exchange resin. In some embodiments, the resin is packed into a column. In particular embodiments, the resin is strong anion-exchange resin such as Q Sepharose™ or QAE Sephadex®. In particular embodiments, the column is a liquid chromatography column.

Liquid chromatography is a process of selectively retaining one or more components of a fluid solution as the fluid solution (mobile phase) permeates through a column of a finely divided substance (stationary phase) by capillary action. The retention of selective components in the fluid solution by the stationary phase results from the higher affinities of the components for the stationary phase than for the mobile phase. Liquid chromatography as used herein includes, but is not limited to, high-performance liquid chromatography (HPLC), ultra-high-performance liquid chromatography (UHPLC), fast protein liquid chromatography (FPLC), and ion exchange chromatography (IEX). Analytes of interest may be retained by the stationary phase and subsequently eluted, or may flow through the stationary phase without being retained. Analytes in the eluate or the effluent may be monitored by a variety of means (e.g., UV, fluorescence, light scattering, or electrical conductivity) based on retention time, peak intensity, and peak area. Further detailed analysis of the analytes may be performed with techniques known in the art, such as, but not limited to, mass spectrometry.

Other types of non-affinity binding resins and columns known in the art may be contemplated for use in the methods provided herein.

By way of example, the diluted CDN mixture may be incubated with Q Sepharose™ resin for a period of about 1 to about 10 hours, e.g., about 3, 4, 5, 6, or more hours, at room temperature on a stirrer; the volume of resin may be equal or similar to the volume of the pre-diluted CDN mixture. The diluted CDN mixture and resin are optionally filtered (e.g., by using a 0.2 M filter). The resin then is packaged into a column and washed using a neutral buffer (e.g., 20 mM Tris-HCl, pH 7.2). CDN is eluted from the column with a elution buffer (e.g., neutral buffer used above plus 100-200 mM NaCl). The elution may be performed for a period of about 1 to about 10 hours, e.g., about 3, 4, 5, 6, or more hours, at room temperature on a stirrer. The CDN eluate may optionally be filtered again (e.g., by a 0.2 μM filter). Other salt buffer compositions, pH, concentration ranges, and parameters, are contemplated for use according to the methods provided herein.

### F. Further Purification of CDN

In some embodiments, the CDN eluate may be concentrated and further purified using nanofiltration. The nanofiltration may be, for example, tangential flow filtration (TFF). In TFF, the sample flows parallel to the filter/membrane and particles that are smaller than the pore size are pushed through as the filtrate while the remainder (retentate) is recycled back to a reservoir. The speed of TFF may be fast, at, e.g., 100 mL/min. In other embodiments, the nanofiltration used is direct flow filtration (DFF). In DFF, the sample flows perpendicular to the membrane face and attempts to pass 100% of the fluid through the membrane.

In some embodiments, CDN is concentrated to at least 5 mg/mL. For example, the CDN is concentrated to at least 10 mg/mL, 15 mg/mL, 20 mg/mL, 25 mg/mL, 30 mg/mL, 35 mg/mL, 35 mg/mL, 40 mg/mL, 45 mg/mL, or 50 mg/mL. In particular embodiments, the CDN is concentrated to 10 mg/mL.

Impurities in the CDN preparation (e.g., plasmid DNA, genomic DNA, proteins, endotoxin, etc.) may be detected by using means readily available in the art such as real-time PCR (qPCR—to test for pDNA and gDNA), NanoOrange® Protein Quantitation Kit (to test for protein presence), or Limulus amebocyte lysate (LAL) assay (to test for the presence of endotoxin). In some embodiments, endotoxin is removed by filtration to a level of less than 10 endotoxin unit (EU).

The purified and concentrated CDN preparation may then be refrigerated or frozen. The preparation may also be lyophilized.

### II. Methods of Use and Related Pharmaceutical Compositions

In some embodiments, the purified CDN (e.g., 2′,3′-cGAMP) may be used as a pharmaceutical product (e.g., adjuvant to a vaccine, a therapeutic compound, or an immunotherapy) for the stimulation of the immune system in a subject in a subject in need of. In other embodiments, the purified CDN (e.g., 2′,3′-cGAMP) is used as a stand-alone treatment to stimulate the immune system in a subject in need thereof. The subject may be a mammal such as a human and may have an infectious disease or cancer.

Examples of pathogenic viruses causing infections include HIV, hepatitis (A. B, or C) virus, herpes virus (e.g., VZV, HSV-1, HAV-6, HSV-II, CMV, Epstein-Barr virus), influenza virus, flaviviruses, echovirus, rhinovirus, coxsackie virus, coronavirus (e.g., SARS-CoV-2), respiratory syncytial virus, mumps virus, rotavirus, measles virus, rubella virus, parvovirus, vaccinia virus, HTLV, dengue virus, papillomavirus, molluscum virus, poliovirus, rabies virus, JC virus, and arboviral encephalitis virus.

Examples of pathogenic bacteria causing infections include chlamydia, rickettsial bacteria, mycobacteria, staphylococci, streptococci, pneumococci, meningococci and gonococci, Klebsiella, proteus, serratia, pseudomonas, legionella, diphtheria, salmonella, bacilli, cholera, tetanus, botulism, anthrax, plague, leptospirosis, and Lyme disease bacteria.

Examples of pathogenic fungi causing include Candida (albicans, krusei, glabrata, tropicalis, etc.), Cryptococcus neoformans, Aspergillus (funigatus, niger, etc.), Genus Mucorales (mucor, absidia, rhizophus), Sporothrix schenkii, Blastomyces dermatitidis, Paracoccidioides brasiliensis, Coccidioides immitis and Histoplasma capsulatum.

Examples of pathogenic parasites causing infections include Entamoeba histolytica, Balantidium coli, Naegleria fowleri, Acanthamoeba sp., Giardia lamblia, Cryptosporidium sp., Pneumocystis, carinii, Plasmodium vivax, Babesia microti, Trypanosoma brucei, Trypanosoma cruzi, Leishmania donovani, Toxoplasma gondii, and Nippostrongylus brasiliensis.

Examples of cancers for treatment include melanoma (e.g., metastatic malignant melanoma), renal cancer (e.g., clear cell carcinoma), prostate cancer (e.g. hormone refractory prostate adenocarcinoma), breast cancer, colon cancer, pancreatic cancer, lung cancer (e.g. non-small cell lung cancer), uterine cancer, ovarian cancer, rectal cancer, cancer of the anal region, stomach cancer, testicular cancer, lymphoma (e.g., non-Hodgkin's lymphoma).

An effective amount of CDN may be used in combination with a prophylactic or therapeutic agent (e.g., vaccine or immunotherapy) to treat these conditions. An “effective” amount refers to the amount of CDN referred to herein that, when used in combination with a therapeutic, relieves one or more symptoms of the treated condition. This amount may vary based on the condition or patient being treated, and can be determined by a healthcare professional using well established principles.

The appropriate dosage level of the pharmaceutical composition described herein may be determined on the basis of a variety of factors, including the patient's age, weight, disease condition, general health, and medical history, as well as the route and frequency of the administration, the pharmacodynamics, and pharmacokinetics of the active ingredient in the composition, and any other drugs that the patient may be taking concurrently.

The pharmaceutical composition may be administered intravenously, intramuscularly, subcutaneously, topically, or any other route of administration that is appropriate for the condition and the drug formulation. In some embodiments, the pharmaceutical composition is administered intranasally.

### III. Exemplary Embodiments

Non-limiting exemplary embodiments of the present disclosure are shown as follows for illustrative purposes.
1. A method of producing highly pure cyclic dinucleotides (CDNs), comprising:
   (a) incubating CDN-producing recombinant E. coli cells in a fermenter for a period of time sufficient to produce a desired amount of CDN;
   (b) reducing the salt concentration of the cell culture of step through dilution;
   (c) isolating CDN from the mixture of step (b) through a column that does not comprise affinity binding; and
   (d) purifying the isolated CDN through nanofiltration.
2. The method of embodiment 1, wherein the E. coli cells are incubated in a minimal fermentation medium in the fermenter.
3. The method of embodiment 2, wherein the minimal fermentation medium comprises:
   i) M9 minimal salts;
   ii) 0.8-3% glucose;
   iii) 5-10 mM MgSO4;
   iv) 0.1-0.3 mM CaCl2); and
   v) 0.01-0.03 mM ferrous sulfate.
4. The method of any one of the preceding embodiments, wherein the recombinant E. coli cells comprise a codon-optimized version of a full-length mouse cyclic guanosine monophosphate (GMP)-adenosine monophosphate (AMP) synthase (mcGAS) gene.
5. The method of any one of the preceding embodiments, wherein the E. coli cells have one or more of the following characteristics:
   contain extra copies of rare E. coli argU, ileY, leuW, proL tRNA genes which corrects for codon bias and dramatically improves expression of sequences from other organisms;
   induce high-level protein expression from promoters with tight control over leaky expression;
   contain a plasmid bearing a constitutive lacI repressor gene, which provides tight control over promoters containing the lacO operator; and
   achieve high efficiency transformations.
6. The method of embodiment 4 or 5, wherein said mcGAS is expressed as a SUMO fusion protein in the E. coli strain.
7. The method of any one of the preceding embodiments, wherein the E. coli cells are grown for about 56 hours in the fermenter to produce CDN.
8. The method of any one of the preceding embodiments, wherein the fermenter has a culture volume of 1 L to 1000 L.
9. The method of any one of the preceding embodiments, wherein between steps (a) and (b) the method comprises:
   collecting the cell culture medium by removing the cultured E. coli cells;
   combining the collected cell culture media comprising CDN; and
   filtering the media to obtain a filtered media comprising CDN.
10. The method of embodiment 9, wherein between the combining step and the filtering step, the method comprises freezing and thawing said cell-free media containing CDN.
11. The method of any one of the preceding embodiments, wherein the CDN is 2′3′-cGAMP.
12. The method of any one of embodiments 1-8, wherein between steps (a) and (b) the method comprises:
   lysing the cultured cells and removing cell debris to obtain a cell-free media with CDN; and
   filtering the media/supernatant obtained following the lysing step to obtain a filtered media comprising CDN.
13. The method of embodiment 11, wherein between the lysing step and the filtering step, the method comprises freezing and thawing said cell-free mixture.
14. The method of embodiment 12 or 13, wherein the CDN is c-di-GMP or 3′,3′-cGAMP.
15. The method of any one of embodiments 9-14, wherein said filtering comprises using a 0.2 μm filter.
16. The method of any one of the preceding embodiments, wherein between steps (b) and (c) the method optionally comprises subjecting the diluted mixture through a weak anion-exchange resin or filtration step to remove impurities.
17. The method of embodiment 16, wherein said filtration uses a 1 kilodalton (kDa) or a 3 kDa cut-off filter.
18. The method of any one of the preceding embodiments, wherein step (c) comprises:
   contacting the diluted mixture containing CDN with a solid support comprising a strong anion-exchange resin, wherein CDN in the mixture binds to the solid support;
   washing the solid support with a washing solution to remove biological materials other than bound CDN, and
   preferentially eluting the bound CDN from the solid support with an elution solution to obtain a substantially pure CDN sample.
19. The method of embodiment 18, wherein the strong anion-exchange resin is Q Sepharose™ or QAE Sephadex®.
20. The method of embodiment 18 or 19, wherein the washing solution comprises a neutral buffer.
21. The method of any one of embodiments 18-20, wherein the washing solution is 20 mM Tris-HCl, pH 7.2.
22. The method of any one of embodiments 18-21, wherein the elution solution comprises 20 mM Tris-HCl, pH 7.2, 100-200 mM NaCl.
23. The method of any one of embodiments 18-22, wherein the substantially pure CDN comprises less than 10% impurities.
24. The method of any one of the preceding embodiments, wherein step (d) further comprises concentrating said CDN using tangential flow nanofiltration.
25. The method of any one of embodiments 1-23, wherein step (d) further comprises concentrating said CDN using direct flow nanofiltration.
26. The method any one of embodiments 18-25, wherein the CDN sample is filtered to remove endotoxin.
27. A pharmaceutical composition comprising a purified CDN obtained from the method of any one of the preceding embodiments.
28. A method of stimulating the immune system in a subject, comprising administering to the subject in need an effective amount of the pharmaceutical composition of embodiment 27.
29. The pharmaceutical composition of embodiment 27 for use in a method of stimulating the immune system in a subject in need thereof.
30. Use of a purified CDN obtained from the process of any one of embodiments 1-26 in the manufacture of a medicament for use in a method of stimulating the immune system in a subject.
31. The method, pharmaceutical composition for use, or use of any one of embodiments 28-30, wherein the immune response stimulation treats a disease, cancer, or infection in the subject.
32. The method, pharmaceutical composition for use, or use of any one of embodiments 28-30, wherein the pharmaceutical composition or medicament is administered in combination with, or is for use in combination with a vaccine or therapeutic compound to stimulate an immune response in the subject.
33. The method, pharmaceutical composition for use, or use of any one of embodiments 28-30, wherein the pharmaceutical composition or medicament is administered or is for use as an adjuvant in antiviral vaccines or cancer immunotherapy.
34. The method, pharmaceutical composition for use, or use of any one of embodiments 28-33, wherein the pharmaceutical composition or medicament is administered intranasally.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art. As used herein, the term “approximately” or “about” as applied to one or more values of interest refers to a value that is similar to a stated reference value. In certain embodiments, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context.

According to the present disclosure, back-references in the dependent claims are meant as short-hand writing for a direct and unambiguous disclosure of each and every combination of claims that is indicated by the back-reference. Any compound disclosed herein can be used in any of the treatment methods here, wherein the individual to be treated is as defined anywhere herein.

In order that this invention may be better understood, the following examples are set forth. These examples are for purposes of illustration only and are not to be construed as limiting the scope of the invention in any manner.

### EXAMPLES

### Example 1: CDN Production Process

Plasmid Construction and Transformation, and Production/Secretion of 2′,3′-cGAMP

Plasmids were constructed and recombinant E. coli cells were generated in a similar fashion as described in Lv et al., ibid.

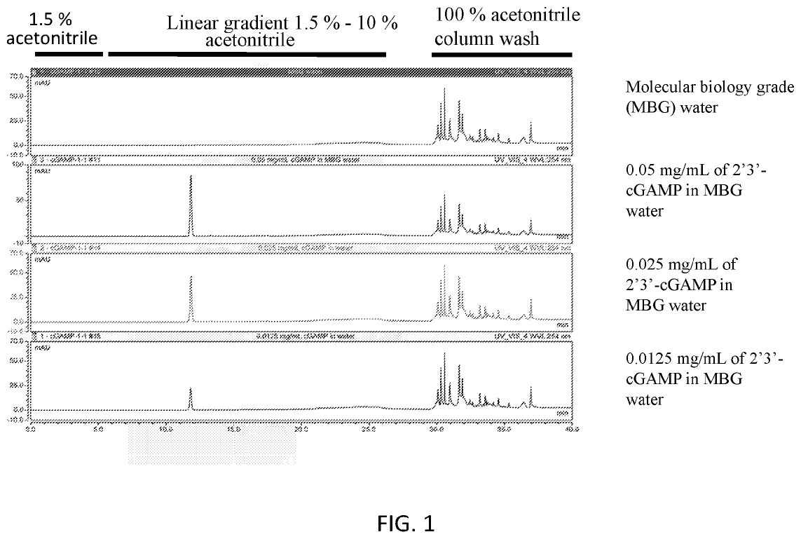

To validate the HPLC results with respect to the detection of 2′,3′-cGAMP disclosed in Lv et al., ibid, we performed HPLC analysis of a chemically synthesized 2′,3′-cGAMP standard in water using the same conditions published therein (FIG. 1). The HPLC system used was an ultra-high-performance liquid chromatography (UHPLC) system. Briefly, 10 μL of 2′,3′-cGAMP sample in 5 mM ammonium acetate, pH 5.0, was injected into a YMC-Pack Pro C18 5 μm (4.6/250) HPLC column in 30° C. and analyte was run at a rate of 1 mL/min. The run was started with 1.5% acetonitrile for 5 min, then a linear gradient of 1.5%-10% acetonitrile was applied for 20 min, followed by a wash with 100% acetonitrile for 10 min. Analyte detection was measured at 254 nm. The control run, without sample is shown in FIG. 1, top panel. In the remaining panels of FIG. 1, the sample was injected into the column and serial dilutions (2×) were performed. These panels showed the correct elution time for 2′,3′-cGAMP which was detected as a distinct single UV absorbance peak. The serial dilutions corresponded to a 2× decrease in the 2′,3′-cGAMP signal (FIG. 1).

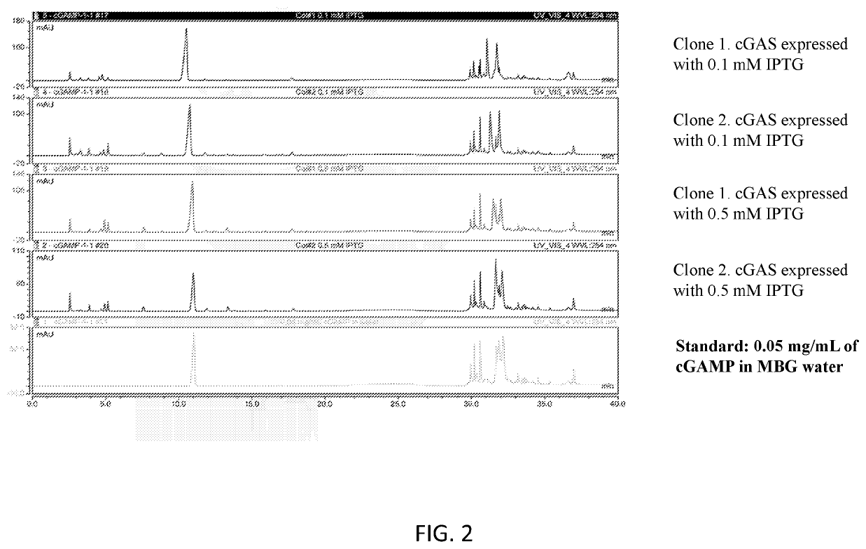

To assess whether 2′,3′-cGAMP was being produced and secreted into the culture medium by recombinant E. coli cells, HPLC analysis of supernatants of two different E. coli clones was performed after shake flask growth in M9 minimal medium (100 mL, 200 rpm, 24 h, 37° C.). The supernatant was collected following IPTG induction using two different IPTG concentrations (FIG. 2, top four panels). The supernatants were analyzed using the same HPLC conditions described above. The results show the presence of the 2′,3′-cGAMP peak, which matched the control (FIG. 2, bottom panel). An expected yield of >0.1 mg/mL is achieved. The presence of 2′,3′-cGAMP was confirmed using MS/MS mass spectrometry.

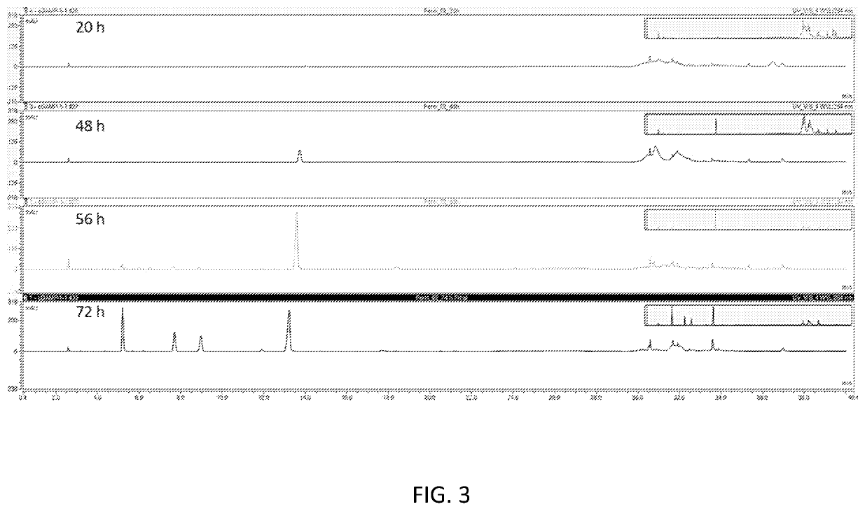

In addition, a fermentation trial was carried out to assess the maximum yield of 2′,3′-cGAMP secreted by the E. coli into the media. Murine cGAS-expressing E. coli cells were incubated in 8 L of M9 medium in a BioFlo® 310 fermenter with constant mixing, dissolved oxygen and pH control at 7.2. The peak representing 2′,3′-cGAMP began to appear at around 48 hours and maxed out at approximately 56 hours, indicating that the maximum yield had been reached then (FIG. 3).

Downstream Processing of 2′,3′-cGAMP

Freeze/thaw cycles of the culture medium containing 2′,3′-cGAMP were performed, followed by 0.2 μM filtration. The medium was then diluted 5-10× with water to reduce salt concentration. At this point, an optional step of subjecting the diluted sample mixture to DEAE chromatography (2′,3′-cGAMP does not bind) was performed to remove impurities. Briefly, bulk adsorption was performed with 100 mL of wet settled resin (EMD) per 1000 mL of diluted medium. Incubation was carried out for 3 hours at room temperature on a stirrer, and the suspension was filtered using a 0.2 μM filter. As an alternative to the DEAE step, we used a molecular weight cut-off (MWCO) filter (3 kDa) to remove impurities from the diluted sample mixture. 2′,3′-cGAMP has a molecular weight of about 674.4 Da.

The diluted sample mixture was then subjected to Q Sepharose™ chromatography. Briefly, bulk adsorption was performed with 100 mL of wet settled resin per 1000 mL of diluted medium. Incubation was carried out for 3 hours at room temperature on a stirrer, and the suspension was then filtered using a 0.2 μM filter. The Q Sepharose™ resin was extensively washed with 20 mM Tris-HCl, pH 7.2, eluted with 20 mM Tris-HCl, pH 7.2, 100-200 mM NaCl in bulk for 3 hours at room temperature on a stirrer, and filtered using a 0.2 μM filter.

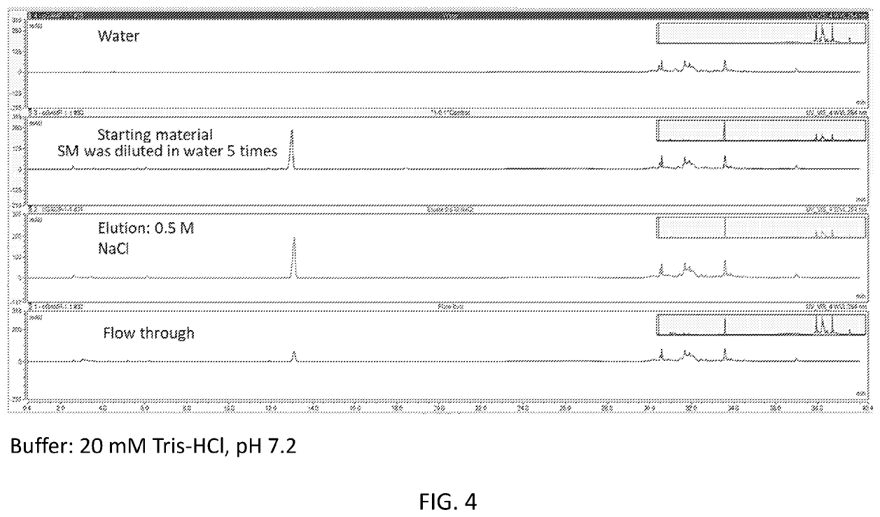

FIG. 4 shows a purification trial after shake flask growth. In this trial, the resulting mixture (referred to as starting material “SM”) was diluted in water 5×, and was then applied to Q Sepharose™ column, washed, and eluted. A flow-through step was carried out to confirm that most of the analyte (2′,3′-cGAMP) was retained (FIG. 4, bottom panel). Indeed, most was retained, with only about 10% cGMAP flowing through.

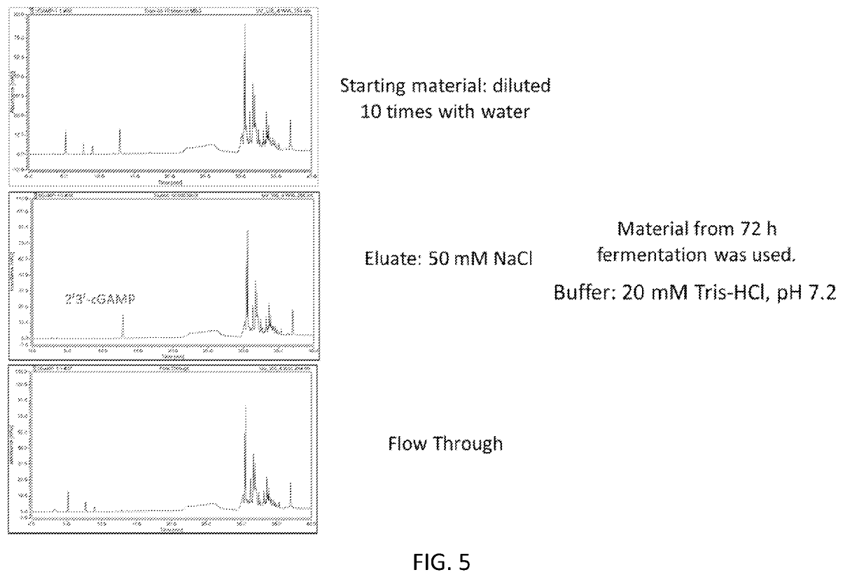

FIG. 5 shows a purification trial after fermentation, the resulting mixture (referred to as starting material “SM”) was diluted in water 10×, and was then applied to Q Sepharose™ column, washed, and eluted. The eluate was 50 mM NaCl using 20 mM Tris-HCl buffer at pH 7.2. A flow-through step was carried out to confirm that most of the analyte was retained (FIG. 5, bottom panel), and indeed, the analyte 2′,3′-cGMAP was retained and did not show up in the flow-through, while the rest of the contaminants showed up in the flow-through.

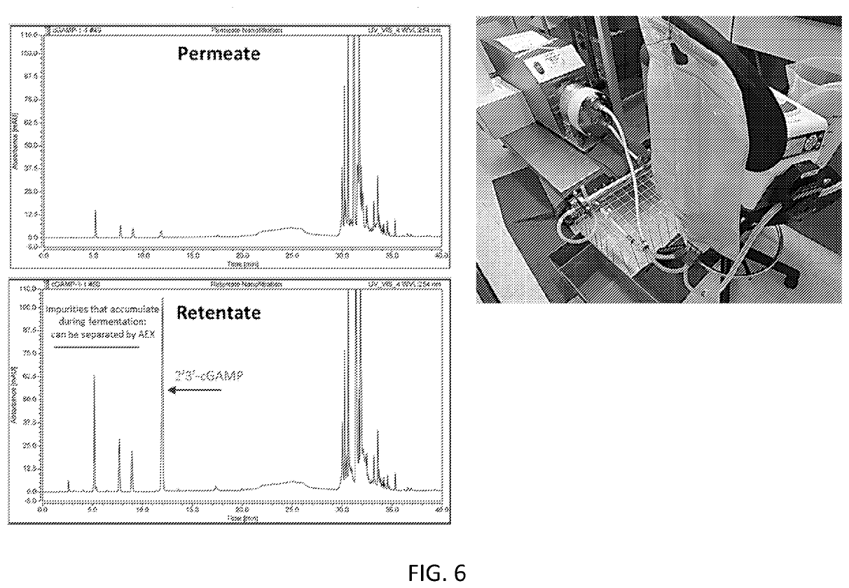

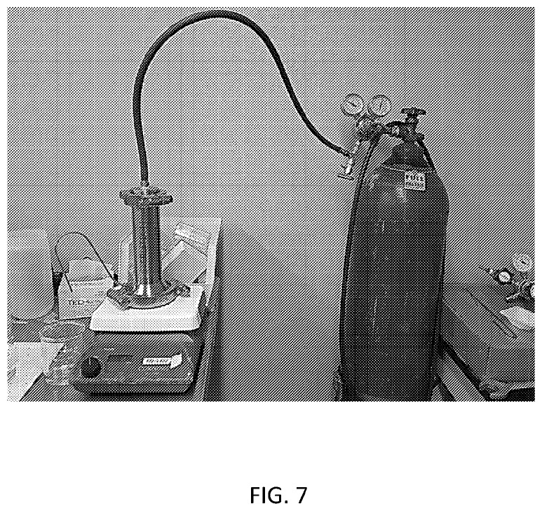

The resulting solution after the above-described steps contained substantially pure 2′,3′-cGAMP. The substantially pure 2′,3′-cGAMP sample solution was then concentrated using tangential flow nanofiltration (FIG. 6) or direct flow nanofiltration (FIG. 7). Briefly, the sample mixture was filtered using a 0.2 μm filter prior to tangential flow nanofiltration. A spiral-wound TFF assembly was used, together with a TFC 600-800 Da sanitary nanofiltration membrane, and a diaphragm pump with disposable head (1000 r/min, room temp). We started nanofiltration with a starting volume of 4 L and ended with 1 L in volume of retentate. Direct flow nanofiltration was performed using a N₂ pressurized unit with the TFC 300-500 Da membrane at 250 PSI at a rate of 0.5 ml/min. The concentrated sample mixture was then sterilized by using 0.2 μm filtration, and was then stored at −20° C. No genomic DNA or plasmid DNA was detected after performing real-time PCR (qPCR). The residual protein measured by NanoOrange® was detected as <10%. 2′,3′-cGAMP concentration was determined by UV. HPLC detection of 2′,3′-cGAMP was confirmed by mass spectrometry. The concentrated 2′,3′-cGAMP solution was tested for and passed a bioburden test.

In conclusion, about 140 mg/L 2′,3′-cGAMP were produced during a 10 L fermentation and around 10 mg of 2′,3′-cGAMP was purified using this method from 4 L of medium.

1. A method of producing cyclic dinucleotides (CDNs), comprising: (a) incubating CDN-producing recombinant E. coli cells in a culture medium in a fermenter to produce a desired amount of CDN; (b) removing the cells from the cell culture medium to obtain a cell-free culture medium containing CDN; (c) filtering the cell-free culture medium to remove cell debris; (d) reducing the salt concentration of the filtered culture medium through dilution; (e) isolating CDN from the mixture of step (d) through a column that does not comprise affinity binding; and (f) purifying the isolated CDN through nanofiltration.

2. The method of claim 1, wherein the recombinant E. coli cells are incubated in a minimal fermentation medium in the fermenter, optionally wherein the minimal fermentation medium is supplemented.

3. The method of claim 2, wherein the supplemented minimal fermentation medium comprises: M9 minimal salts; 0.8-3% glucose; 5-10 mM MgSO 4; 0.1-0.3 mM CaCl 2; and 0.01-0.03 mM ferrous sulfate.

4. The method of claim 1, wherein the recombinant E. coli cells comprise a codon-optimized version of a mouse cyclic guanosine monophosphate (GMP)-adenosine monophosphate (AMP) synthase (mcGAS) gene.

5. The method of claim 1, wherein the recombinant E. coli cells contain (i) extra copies of one or more of E. coli argU, ileY, leuW, and proL tRNA genes; and/or (ii) a plasmid bearing a constitutively active lacI repressor gene.

6. The method of claim 4, wherein the mcGAS gene is expressed in the recombinant E. coli cells as part of a small ubiquitin-like modifier (SUMO) fusion protein.

7. (canceled)

8. The method of claim 1, wherein the fermenter has a culture volume of 1 L to 1000 L.

9. (canceled)

10. (canceled)

11. (canceled)

12. The method of claim 1, wherein between steps (d) and (e) the method comprises subjecting the diluted mixture through weak anion-exchange resin or molecular weight cut-off (MWCO) filtration to remove impurities.

13. (canceled)

14. The method of claim 1, wherein step (e) comprises: contacting the diluted mixture containing CDN with a solid support comprising strong anion-exchange resin, wherein CDN in the mixture binds to the solid support; washing the solid support with a washing buffer to remove biological materials other than bound CDN, and preferentially eluting the bound CDN from the solid support with an elution buffer to obtain a substantially pure CDN preparation.

15. (canceled)

16. The method of claim 14, wherein the washing buffer is a neutral buffer, optionally comprising 20 mM Tris-HCl, pH 7.2.

17. The method of claim 14, wherein the elution buffer comprises 20 mM Tris-HCl, pH 7.2, and 100-200 mM NaCl.

18. The method claim 14, wherein the CDN preparation is further filtered to remove endotoxin.

19. The method of claim 1, wherein step (f) comprises concentrating the CDN using tangential flow nanofiltration or direct flow nanofiltration.

20. The method of claim 1, wherein the CDN is 2′,3′-cGAMP; c-di-GMP; or 3′,3′-cGAMP.

21. A pharmaceutical composition comprising a purified CDN obtained by the method of claim 1.

22. A method of stimulating the immune system in a subject in need thereof, comprising administering to the subject an effective amount of the pharmaceutical composition of claim 21.

23.-25. (canceled)
